(19) United States
(12) Patent Application Publication
(10) Pub. No.: US 2023/0110297 A1
(43) Pub. Date: Apr. 13, 2023

(54) COMPOSITIONS FOR TREATMENT OF PSORIASIS OF THE SCALP

(71) Applicant: Innocan Pharma Ltd., Herzliya (IL)

(72) Inventor: Nir AVRAM, Metar (IL)

(73) Assignee: Innocan Pharma Ltd., Herzliya (IL)

(21) Appl. No.: 17/907,262

(22) PCT Filed: May 19, 2021
(86) PCT No.: PCT/IL2021/050570

§ 371 Date: Sep. 25, 2022

Related U.S. Application Data
(60) Provisional application No. 63/029,627, filed on May 25, 2020.

(51) Int. Cl. A61K 31/05; A61K 31/60; A61K 35/04; A61K 45/06; A61K 9/70

(52) U.S. Cl. CPC A61K 31/05; A61K 31/60; A61K 35/04; A61K 45/06; A61K 9/7015

(57) ABSTRACT

Described herein are compositions for topical administration, to relieve psoriasis of the scalp. The compositions comprise a cannabinoid and at least one additional active ingredient. The additional active ingredient may be salicylic acid or coal tar. The compositions may be biphasic compositions comprising an aqueous phase and an oil-based phase. The two phases may be contained in a single container and mixed, for example, by shaking, before administration of the composition to the scalp of the patient.

### CROSS REFERENCE TO RELATED APPLICATIONS

Benefit is claimed to U.S. Provisional Patent Application 63/029,627 filed May 25, 2020; the contents of which is incorporated by reference herein in its entirety.

### FIELD

Provided herein are topical compositions for treatment of scalp psoriasis.

### BACKGROUND

Psoriasis of the scalp is a skin disorder associated with any of the following symptoms: dry scalp, scaly patches on the scalp, hair loss, burning, soreness and white scales. Psoriasis of the scalp may cover a few spots or large areas of the scalp, extending from the forehead to the back of the neck and around the ears. Scalp psoriasis may be difficult to treat. Psoriasis of the scalp may be treated with intralesional steroids injected to the affected areas, but such treatment is typically used in small areas of the scalp. In addition, steroid use is often associated with side effects.

### SUMMARY

Described herein are compositions for topical administration, to relieve psoriasis of the scalp. The compositions comprise a cannabinoid and at least one additional active ingredient. The additional active ingredient may be salicylic acid or coal tar. The compositions may be biphasic compositions comprising an aqueous phase and an oil-based phase. The two phases may be contained in a single container and mixed, for example, by shaking, before administration of the composition to the scalp of the patient.

Additionally described herein are methods for treating psoriasis of the scalp comprising administering to a person in need thereof a composition comprising a pharmaceutically effective amount of a cannabinoid and of at least one additional active ingredient.

The foregoing and other objects, features, and advantages will become more apparent from the following detailed description.

### DETAILED DESCRIPTION

Unless otherwise noted, technical terms are used according to conventional usage. Definitions of common terms in pharmaceutical sciences can be found in Troy et al. Remington: The Science and Practice of Pharmacy. Published by Lippincott Williams & Wilkins, 2006. In case of conflict, the present specification, including explanations of terms, will control. In addition, all the materials, methods, and examples are illustrative and not intended to be limiting.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

Definitions:

Allantoin: (2,5-Dioxo-4-imidazolidinyl) urea.

Cannabinoid: A cannabinoid is a chemical compound that acts on cannabinoid receptors in cells in mammals, including in humans. Cannabinoids can be manufactured synthetically or obtained from various parts of the genus Cannabis, in particular, from the species Cannabis Sativa. Cannabinoids from the cannabis plant are referred to as phytocannabinoids. Two preferred cannabinoids according to various embodiments, are (−)-trans-Δ⁹-tetrahydrocannabinol, and/or isomers thereof (THC) and cannabidiol (CBD). Alternatively, a cannabinoid may be in the form of cannabis extract. Alternatively, a cannabinoid may be in the form of a synthetic cannabinoid. Compositions described herein may comprise one cannabinoid or multiple cannabinoids, such as a combination of CBD and THC.

Salicylic Acid: 2-hydroxybenzoic acid.

Zinc-PCA: Proline, 5-oxo-, zinc salt. Zinc pidolate.

### Description of Embodiments

Topical compositions for treating psoriasis of the scalp are commercially available in “wash off” form, which is intended to be washed out within minutes of application, and in “leave on” form, which is intended to be applied and left on the scalp for many hours. In general, compositions which are formulated for leaving on the scalp are typically more effective than “wash off” compositions in treating psoriasis of the scalp. However, if “leave on” compositions are left on the scalp for long periods of time, they may cause irritation of the scalp. Some “leave on” compositions comprise alcohol, which has been found to cause dryness of the skin and irritation, which may be especially problematic in patients suffering from psoriasis.

Some compositions for treating psoriasis which are intended to be left on the scalp comprise significant amounts of oil-based solvent. Such compositions, when applied to the scalp, may provide an unaesthetic, oily appearance to the hair. as a result, treatment may be limited to nighttime, before sleep.

Described herein, according to some embodiments, are compositions which are intended to be left on the scalp or to be washed off, comprising a cannabinoid and at least one additional active ingredient. The compositions comprise biphasic mixtures, having an aqueous phase and an oil-based phase, which are packaged together in the same receptacle, but exist primarily as two separate phases. When the composition is shaken, the phases combine, as determined by visual examination, and can then be applied to an affected area of a patient suffering from psoriasis of the scalp. It is suggested that such compositions will be advantageous relative to currently available compositions by providing effective treatment without providing an unaesthetic appearance and without causing irritation to the scalp.

Compositions according to embodiments described herein may comprise a cannabinoid or a plurality of cannabinoids selected from the group consisting of CBD and THC. Optionally, the cannabinoid is CBD. The cannabinoid may be present in an amount of between 0.001 and 10%, preferably 0.1 and 2 percent, by weight, of the compositions. Optionally, the cannabinoid is present in an amount of 0.25% by weight.

According to an embodiment, the additional active ingredient is salicylic acid. The salicylic acid may be present in the composition in an amount between 1.8% and 3.0% by weight. Optionally, the salicylic acid is present in the composition in an amount of 2.0%.

According to an embodiment, the additional active ingredient is coal tar. Optionally, the coal tar is present in the composition in an amount of 0.5-5% by weight.

According to an embodiment, the composition comprises hemp oil in the oil phase of the composition. Optionally, hemp oil is present in the composition in an amount of between 0.5% and 5% of the composition. Optionally, hemp oil is present in an amount of 1% of the composition.

According to an embodiment, the composition has less than 1% alcohol by weight. According to an embodiment, the composition is free of C1-C4 alcohol. For example, the composition is free of ethanol and isopropanol.

Optionally the composition is free of a steroid.

According to an embodiment the ratio, by weight of aqueous to non-aqueous phase to oil-based phase is between 60:40 to 90:10.

According to an embodiment, the composition comprises less than 5% by weight of emulsifier, optionally, the composition is free of emulsifier.

According to an embodiment, the composition comprises a solubilizer. The solubilizer may be polysorbate-20.

According to an embodiment, the composition comprises at least one additional ingredient selected from the group consisting of: menthol, tea tree oil, Zinc-PCA, allantoin, and olive oil.

According to an embodiment, the composition comprises at least one additional active agent selected from the group consisting of methotrexate, cyclosporine, hydroxycarbamide, fumaric acid esters, a retinoid, efalizumab and alefacept, vitamin D and derivatives thereof, calcipotriene, betamethasone, halobetasol and Metaderm®. Metaderm ® is a plant based lotion or cream which comprises the following plant extracts: achillea millefolium, aesculus hippocastanum, berberis vulgaris, conium maculatum, matricaria chamomilla, phytolacca decandra, rhus toxicodendron, and sanguinaria canadensis. Optionally, the fumaric acid ester comprises dimethylfumarate. Optionally, the retinoid is tazarotene or acitretin.

Optionally, the menthol is present in the composition in an amount between 0.1-1%, preferably 0.2%.

Optionally, the tea tree oil is present in the composition in an amount between 0.1-1%, preferably 0.3%.

Optionally, the Zinc-PCA is present in the composition in an amount between 0.05-0.5%, preferably 0.1%.

Optionally, the allantoin is present in the composition in an amount between 0.1-1%, preferably 0.5%.

Optionally, the olive oil is present in the composition in an amount between 0.5-5%, preferably 1%.

The viscosity of the composition is preferably in the range of between 0.5 and 200 centipoise (cps) at 25° C. Preferably, the determination of viscosity is performed immediately after shaking.

According to an embodiment, the composition may be in the form of a liquid, provided in a bottle for spraying onto an affected area. The bottle may have a flip cap and be used to apply directly to the scalp. Alternatively, the bottle may be a hair-root applicator bottle equipped with a comb at the bottle opening to allow the bottle opening to be adjacent to the scalp when applying. The bottle may be equipped with a dropper to allow easy application to the scalp. Optionally, the composition may be in an aerosol spray or a pump spray. The pump spray may be a bottle equipped with a button spray or a trigger spray. The compositions may be packaged in a packaging and provided with instructions to shake before use.

In addition to active ingredients, compositions described herein may further comprise at least one inert ingredient. The inert ingredient may be selected from the group consisting of: water, a solvent, an emollient, a moisturizer, a pH adjustment agent, a polymer, a humectant, an occlusive agent, a preservative, a thickener, an anti-irritation agent, a conditioning agent, a buffer, a vitamin, an extract, a natural oil, a wax, a penetration enhancer, a peptide, a sugar derivative, a fatty acid, a fatty alcohol, a silicone, a polyethyl-glycol, a fragrance, a pigment, an ester, a triglyceride, an antioxidant and an absorbing powder.

According to an embodiment, the composition comprises an aqueous phase and an oil-based, non-aqueous phase. The biphasic composition may be shaken or stirred by the user before administration. Optionally, the non-aqueous phase comprises the cannabinoid. Optionally, upon shaking the composition, both phases mix and do not separate for at least 10 seconds, preferably at least 30 seconds, most preferably at least 2 minutes.

It is suggested that a biphasic composition may provide stability to the composition in that the water-soluble active ingredient is contained in an aqueous environment and the cannabinoid is contained in a non-aqueous environment. Biphasic compositions, when stored, are separated into two phases. Before use, the patient is instructed to shake the container in which the composition is stored and from which the composition is dispensed for a number of seconds between 2 and 5 times. The phases of the biphasic composition then combine, as determined by visual inspection, and the composition can be applied, preferably by spraying, onto the scalp of the patient in need.

Phase separation optionally happens in greater than 30 seconds from the shaking of the biphasic composition. Phase separation preferably happens between 30 sections from the shaking of the biphasic composition until 10 minutes from the shaking of the biphasic composition. Most preferably, phase separation happens between 1 minute from shaking of the biphasic composition until 3 minutes from shaking the biphasic composition.

According to an embodiment, the cannabinoid is dissolved in an oil. Optionally, the oil may be hemp oil, olive oil, or an essential oil. Optionally, the oil is an ester, a triglyceride, a hydrocarbon, a fatty alcohol, mineral oil, silicon oil, ethoxylate alcohol, or vegetable oil.

According to an embodiment, the pH of the composition after shaking is between 3 and 4. Optionally, the pH is 3.2. Optionally, the pH electrode is calibrated at pH of 4 and pH of 7, then immediately immersed in the composition after shaking.

Additional embodiments of the composition relate to a pharmaceutical composition for topical administration comprising a cannabinoid and an additional active ingredient selected from the group consisting of: tazarotene, calcipotriene, betamethasone, halobetasol and Metaderm®.

Additional embodiments relate to methods for treatment of a condition of the scalp comprising administering to a patient in need thereof, a pharmaceutical composition via the topical route comprising a cannabinoid and an additional active ingredient selected from the group consisting of salicylic acid and coal tar. The condition of the scalp is selected from the group consisting of: psoriasis of the scalp, dandruff, and seborrheic dermatitis. The composition may comprise two phases, an aqueous and an oil-based phase.

Treatment of psoriasis of the scalp may involve treatment of symptoms of psoriasis including one or more than one of the following symptoms: thickened, dry, scaly, flaky, cracked, itchy, red and or inflamed skin.

Treatment of dandruff may involve treatment of symptoms of dandruff, including one or more than one of the following symptoms: flaking and itching of the scalp.

According to an embodiment, the composition may be applied daily, optionally, between 1 and 4 times daily. Optionally, the composition is applied and left on the patient's scalp.

According to an embodiment, the composition may be applied in an amount of between about 0.05 milliliter (ml) and about 1 ml per spray (per pump release). Optionally, the composition may be applied in an amount between 0.1 ml and 0.25 ml per spray. The composition may be applied via a single spray burst or multiple spray bursts to cover an affected area.

The following examples are provided to illustrate certain particular features and/or embodiments. These examples should not be construed to limit the disclosure to the particular features or embodiments described.

### EXAMPLES

### Example 1

A spray for treatment of psoriasis of the scalp was prepared using the ingredients described in table 1:

TABLE 1

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Zinc-PCA | Anti-inflammatory | 0.10 |
| Propylene glycol | Carrier and humectant | 2.0 |
| Menthol | Active agent | 0.20 |
| Octyldodecanol | Emollient & solvent | 15.0 |
| Olive Oil | Emollient | 1.0 |
| Salicylic Acid | Active agent | 2.0 |
| BHT | Antioxidant | 0.05 |
| Sativa seed (Hemp) oil | Solvent | 1.0 |
| Cannabidiol | Active agent | 0.25 |
| Tea tree oil | Essential oil antifungal | 0.3 |
| | agent | |
| Polysorbate-20 | Solubilizer | 2.0 |
| Sodium Hydroxide | pH adjusting agent | 0.3 |

In a main vessel, water, disodium EDTA, allantoin and zinc-PCA were added and mixed until completely dissolved. In a separate vessel, menthol and propylene glycol were dissolved, then added to the main vessel while mixing. In a separate vessel, octyldodecanol, olive oil, salicylic acid, BHT, hemp oil, cannabidiol and tea tree oil were added and mixed to form the oil-based phase. The oil-based ingredients were combined with polysorbate-10 (Tween ®-20) and were then added to the main vessel. The pH was checked and adjusted with NaOH to between 3 and 4. The product was then introduced into pump-bottles.

The viscosity of the composition was analyzed and found to be less than 200 cps.

The composition prepared is advantageous in that it comprises an aqueous layer and an oil-based layer. The cannabinoid remains stable while in the oil-based layer. Upon mixing by shaking, the biphasic mixture is mixed to form a single phase for 2 minutes and 45 seconds, allowing a patient in need to apply the composition having a cannabinoid and another active ingredient, conveniently, to the affected area. After administration, much of the water-phase of the composition evaporates and does not leave an oily residue on a patient's hair and scalp. After administration, the aqueous and oil-based layers separate, providing additional stability to the composition.

### Example 2

A spray for treatment of psoriasis of the scalp is prepared using a similar process as in Example 1, using the ingredients in Table 2.

TABLE 2

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Symsoft ® Scalp | Soothing of itching | 1.00 |
| (Trideceth-9, PEG-5- | scalp | |
| Ethylhexanoate, | | |
| Water, Hydroxyphenyl | | |
| Propamidobenzoic Acid) | | |
| Propylene glycol | Carrier and humectant | 3.0 |
| Menthol | Active agent | 0.20 |
| Octyldodecanol | Emollient & solvent | 15.0 |
| Olive Oil | Emollient | 1.0 |
| Salicylic Acid | Active Agent | 2.0 |
| BHT | Antioxidant | 0.05 |
| Sativa seed (Hemp) oil | Solvent | 1.0 |
| Cannabidiol | Active agent | 0.25 |
| Tea tree oil | Essential oil antifungal | 0.3 |
| | agent | |
| Polysorbate-20 | Solubilizer | 2.0 |

Symsoft® Scalp is an clear, light colored liquid made by Symrise and available at www. Symrise.com.

### Example 3

A spray for treatment of psoriasis of the scalp is prepared using a similar process as in Example 1, using the ingredients in Table 3.

TABLE 3

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Zinc-PCA | Anti-inflammatory | 0.10 |
| Propanediol | Humectant | 2.0 |
| Menthol | Active agent | 0.20 |
| Octyldodecanol | Emollient & solvent | 15.0 |
| Olive Oil | Emollient | 1.0 |
| Salicylic Acid | Active agent | 2.0 |
| BHT | Antioxidant | 0.05 |
| Sativa seed (Hemp) oil | Solvent | 1.0 |
| Cannabidiol | Active agent | 0.25 |
| Leptospermum Scoparium | Essential oil antifungal | 0.1 |
| Branch/Leaf Oil | agent | |
| Caprylyl/Capryl Glucoside | Solubilizer | 1.0 |

### Example 4

A spray for treatment of psoriasis of the scalp is prepared using a similar process as in Example 1, using the ingredients in Table 4.

TABLE 4

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Aloe Vera gel | Soothing agent | 2.0 |
| Symsoft Scalp (Trideceth-9, | Soothing of itching | 1.0 |
| PEG-5-Ethylhexanoate, | scalp | |
| Water, Hydroxyphenyl | | |
| Propamidobenzoic Acid) | | |
| Propylene glycol | Carrier and humectant | 3.0 |
| Menthol | Active agent | 0.10 |
| Octyldodecanol | Emollient & solvent | 15.0 |
| Avocado Oil | Emollient | 1.0 |
| Salicylic Acid | Active agent | 2.0 |
| BHT | Antioxidant | 0.05 |
| Sativa seed (Hemp) oil | Solvent | 1.0 |
| Bisabolol | Soothing agent | 0.10 |
| Cannabidiol | Active agent | 0.25 |
| Tea tree oil | Essential oil antifungal | 0.3 |
| | agent | |

### Example 5

A spray for treatment of psoriasis of the scalp is prepared using a similar process as in Example 1, using the ingredients in Table 5.

TABLE 5

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Aloe Vera gel | Soothing agent | 2.0 |
| Symsoft Scalp (Trideceth-9, | Soothing of itching | 1.0 |
| PEG-5-Ethylhexanoate, | scalp | |
| Water, Hydroxyphenyl | | |
| Propamidobenzoic Acid) | | |
| Propylene glycol | Carrier and humectant | 3.0 |
| Menthol | Cooling agent | 0.10 |
| Octyldodecanol | Emollient & solvent | 15.0 |
| Avocado Oil | Emollient | 1.0 |
| Salicylic Acid | Active agent | 2.0 |
| BHT | Antioxidant | 0.05 |
| Sativa seed (Hemp) oil | Solvent | 1.0 |
| Tumeric oil | Anti-inflammatory | 0.10 |
| Cannabidiol | Active agent | 0.25 |
| Tea tree oil | Essential oil antifungal | 0.3 |
| | agent | |
| Caprylyl/Capryl Glucoside | Solubilizer | 1.0 |

### Example 6

A spray for treatment of psoriasis of the scalp is prepared using a similar process as in Example 1, using the ingredients in Table 6.

TABLE 6

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Aloe Vera gel | Soothing agent | 1.0 |
| Zinc-PCA | Anti-inflammatory | 0.10 |
| Propylene glycol | Carrier and humectant | 2.0 |
| Menthol | Cooling agent | 0.20 |
| Octyldodecanol | Emollient & solvent | 15.0 |
| Olive Oil | Emollient | 1.0 |
| Salicylic Acid | Active Agent | 2.0 |
| BHT | Antioxidant | 0.05 |
| Bisabolol | Soothing Agent | 0.1 |
| Sativa seed (Hemp) oil | Solvent | 1.0 |
| Cannabidiol | Active Agent | 0.25 |
| Tea tree oil | Essential oil antifungal | 0.3 |
| | agent | |
| Polysorbate-20 | Solubilizer | 1.0 |
| Sodium Hydroxide | pH adjusting agent, to | 0.4 |
| | pH of 3.5 | |

### Example 7

A spray for treatment of psoriasis of the scalp is prepared using a similar process as in Example 1, using the ingredients in Table 7.

TABLE 7

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Aloe Vera gel | Soothing agent | 1.0 |
| Zinc-PCA | Anti-inflammatory | 0.10 |
| Propylene glycol | Carrier and humectant | 2.0 |
| Menthol | Active agent | 0.20 |
| Octyldodecanol | Emollient & solvent | 20.0 |
| Olive Oil | Emollient | 1.0 |
| Salicylic Acid | Active agent | 3.0 |
| BHT | Antioxidant | 0.05 |
| Bisabolol | Soothing agent | 0.1 |
| Sativa seed (Hemp) oil | Solvent | 1.0 |
| Cannabidiol | Active agent | 0.25 |
| Tea tree oil | Essential oil antifungal | 0.3 |
| | agent | |
| Polysorbate-20 | Solubilizer | 1.0 |
| Sodium Hydroxide | pH adjusting agent, to | 0.4 |
| | pH of 3.5 | |

### Example 8

A spray for treatment of psoriasis of the scalp is prepared using a similar process as in Example 1, using the ingredients in Table 8:

TABLE 8

| | | Amount |
|---|---|---|
| Ingredient | Function | (percent by weight) |
| Water | Aqueous phase solvent | Up to 100 |
| Disodium EDTA | Chelating agent | 0.05 |
| Allantoin | Skin protectant | 0.50 |
| Propylene glycol | Carrier and humectant | 2.0 |
| Menthol | Active agent | 0.20 |
| Octyldodecanol | Emollient & solvent | 15.0 |
| Castor oil | Hair growth promoter | 2.0 |
| Olive Oil | Emollient | 1.0 |
| Salicylic Acid | Active Agent | 3.0 |
| BHT | Antioxidant | 0.05 |
| Bisabolol | Soothing agent | 0.1 |
| Sativa seed (Hemp) oil | Solvent | 1.0 |
| Cannabidiol | Active Agent | 0.25 |
| Coal Tar (Solution | Active Agent | 1.3 |
| comprising 84.3% Coal Tar | | |
| in solvent) | | |

### Example 9

### Suggested Treatment Method

A composition prepared according to any of the previous examples is applied to affected areas one to four times daily or as directed by a doctor.

Before applying, the patient twists the flow-control cap to open. If using after shampooing hair, the patient's hair is towel dried. The composition may be applied from bottle directly to scalp, and massaged in. To avoid running or dripping, the patient should take care not squeeze bottle too hard. the composition should be stored at 20-25° C. (68-77 degrees F.) and protected from humidity.

According to an embodiment, disclosed is a biphasic pharmaceutical composition for treatment of psoriasis comprising an aqueous phase and an oil-based phase, wherein the composition comprises a cannabinoid and at least one additional agent selected from the group consisting of salicylic acid and coal tar. Optionally, the cannabinoid is selected from the group consisting of full spectrum cannabis, CBD and THC. Optionally, the cannabinoid is CBD. Optionally, wherein the cannabinoid is present in an amount between 0.001% and 10% by weight of the composition. Optionally, the cannabinoid is present in an amount between 0.1% and 1% by weight of the composition. Optionally, the cannabinoid is present in an amount of 0.25% by weight. Optionally, the additional agent is salicylic acid. Optionally, the salicylic acid is present in an amount between 1.8% and 3% by weight of the composition. Optionally, the additional agent is coal tar. Optionally, the coal tar is present in an amount between 0.5-5% by weight of the composition. Optionally, the compositions has a viscosity of between 0.5 and 200 centipoise at 25° C. immediately after shaking. Optionally, the composition further comprises at least one of the agents selected from the group consisting of: menthol, tea tree oil, Zinc-PCA, allantoin, olive oil, methotrexate, cyclosporine, hydroxycarbamide, fumaric acid esters, a retinoid, efalizumab and alefacept, vitamin D and derivatives thereof, calcipotriene, betamethasone, halobetasol and a mixture of the following plant extracts: achillea millefolium, aesculus hippocastanum, berberis vulgaris, conium maculatum, matricaria chamomilla, phytolacca decandra, rhus toxicodendron, and sanguinaria canadensis. Optionally, the composition is free of a C1-C4 alcohol. Optionally, the composition is free of a steroid. Optionally, after shaking the composition by hand, the phases do not mix and separate only after between 30 seconds and 10 minutes following shaking. Optionally, after shaking the composition by hand, the phases do not mix and separate only after between 2 and 3 minutes following shaking. Optionally, the composition is for treatment of psoriasis of the scalp.

Some embodiments relate to a method for treatment of a condition of the scalp selected from the group consisting of: psoriasis of the scalp, dandruff and seborrheic dermatitis comprising topically administering to a patient in need thereof, a composition comprising an aqueous phase and an oil-based phase, wherein the composition comprises a cannabinoid and at least one additional agent selected from the group consisting of salicylic acid and coal tar. Optionally, the cannabinoid is selected from the group consisting of full spectrum cannabis, CBD and THC. Optionally, the cannabinoid is CBD. Optionally, the cannabinoid is present in an amount between 0.001% and 10% by weight of the composition. Optionally, the cannabinoid is present in an amount between 0.1% and 1% by weight of the composition. Optionally, the cannabinoid is present in an amount of 0.25% by weight. Optionally, the additional agent is salicylic acid. Optionally, the salicylic acid is present in an amount between 1.8% and 3% by weight of the composition. Optionally, the additional agent is coal tar. Optionally, the coal tar is present in an amount between 0.5-5% by weight of the composition. Optionally, the composition has a viscosity of between 0.5 and 200 centipoise at 25° C. immediately after shaking. Optionally, the composition comprises at least one of the agents selected from the group consisting of: menthol, tea tree oil, Zinc-PCA, allantoin, olive oil, methotrexate, cyclosporine, hydroxycarbamide, fumaric acid esters, a retinoid, efalizumab and alefacept, vitamin D and derivatives thereof, calcipotriene, betamethasone, halobetasol and a mixture of the following plant extracts: achillea millefolium, aesculus hippocastanum, berberis vulgaris, conium maculatum, matricaria chamomilla, phytolacca decandra, rhus toxicodendron, and sanguinaria canadensis. Optionally, the composition is free of a C1-C4 alcohol. Optionally, the composition is free of a steroid. Optionally, after shaking the composition by hand, the phases do not mix and separate only after between 30 seconds and 10 minutes following shaking Optionally, after shaking the composition by hand, the phases do not mix and separate only after between 2 and 3 minutes following shaking. Optionally, the condition is psoriasis of the scalp. Optionally, the treatment is for symptoms of psoriasis including one or more than one of the following symptoms: thickened, dry, scaly, flaky, cracked, itchy, red skin and inflamed skin. Optionally, the composition is administered by spray in an amount of between 0.05 ml and 1 ml per spray. Optionally, the composition is administered between once and 4 times daily. Optionally, the method further comprises shaking the composition before administering the composition topically. Optionally, the composition is administered to the scalp of a patient via a hair root applicator bottle, a dropper, an aerosol spray or pump spray. Optionally, the method further comprises leaving the composition on the scalp for between 1 and 12 hours after application.

Further embodiments relate to packaged, sealed pharmaceutical composition comprising: a composition as described above, contained within a sealed container, the container selected the group consisting of a bottle having a flip cap, a bottle having a root applicator, a bottle having a dropper, an aerosol spray canister, a pump spray canister.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A biphasic pharmaceutical composition for treatment of psoriasis comprising an aqueous phase and an oil-based phase, wherein the composition comprises a cannabinoid and at least one additional agent selected from the group consisting of salicylic acid and coal tar.

2. The composition according to claim 1 wherein the cannabinoid is selected from the group consisting of full spectrum cannabis, CBD and THC.

3. The composition according to claim 2 wherein the cannabinoid is CBD.

4. The composition according to claim 1 wherein the cannabinoid is present in an amount between 0.001% and 10% by weight of the composition.

5. The composition according to claim 4 wherein the cannabinoid is present in an amount between 0.1% and 1% by weight of the composition.

6. The composition according to claim 5 wherein the cannabinoid is present in an amount of 0.25% by weight.

7. The composition according to claim 1 wherein the additional agent is salicylic acid.

8. The composition according claim 7 wherein the salicylic acid is present in an amount between 1.8% and 3% by weight of the composition.

9. The composition according to claim 1 wherein the additional agent is coal tar.

10. The composition according claim 9 wherein the coal tar is present in an amount between 0.5-5% by weight of the composition.

11. The composition according to claim 1 having a viscosity of between 0.5 and 200 centipoise at 25° C. immediately after shaking.

12. The composition according to claim 1 comprising at least one of the agents selected from the group consisting of: menthol, tea tree oil, Zinc-PCA, allantoin, olive oil, methotrexate, cyclosporine, hydroxycarbamide, fumaric acid esters, a retinoid, efalizumab and alefacept, vitamin D and derivatives thereof, calcipotriene, betamethasone, halobetasol and a mixture of the following plant extracts: achillea millefolium, aesculus hippocastanum, berberis vulgaris, conium maculatum, matricaria chamomilla, phytolacca decandra, rhus toxicodendron, and sanguinaria canadensis.

13. The composition according to claim 1, and free of a C1-C4 alcohol.

14. The composition according to claim 1, and free of a steroid.

15. The composition according to claim 1 wherein after shaking the composition by hand, the phases do not mix and separate only after between 30 seconds and 10 minutes following shaking.

16. The composition according to claim 15 wherein after shaking the composition by hand, the phases do not mix and separate only after between 2 and 3 minutes following shaking.

17. (canceled)

18. A method for treatment of a condition of the scalp selected from the group consisting of: psoriasis of the scalp, dandruff and seborrheic dermatitis comprising topically administering to a patient in need thereof, a composition comprising an aqueous phase and an oil-based phase, wherein the composition comprises a cannabinoid and at least one additional agent selected from the group consisting of salicylic acid and coal tar.

19.-35. (canceled)

36. The method according to claim 18 wherein the composition is administered by spray in an amount of between 0.05 ml and 1 ml per spray.

37. The method according to claim 18 wherein the composition is administered between once and 4 times daily.

38. The method according to claim 18 further comprising shaking the composition before administering the composition topically.

39.-41. (canceled)
